(19) United States
(12) Patent Application Publication
(10) Pub. No.: US 2022/0249543 A1
(43) Pub. Date: Aug. 11, 2022

(54) Trona Based Therapeutic Compositions and Use and Manufacture Thereof

(71) Applicant: Moxiyo, LLC, Park City, UT (US)

(72) Inventor: Pete Fuller, Park City, UT (US)

(21) Appl. No.: 17/667,584

(22) Filed: Feb. 9, 2022

Related U.S. Application Data
(60) Provisional application No. 63/147,990, filed on Feb. 10, 2021.

(51) Int. Cl. A61K 33/00; A61K 47/02; A61K 47/12; A61K 9/00; A61K 9/70

(52) U.S. Cl. CPC A61K 33/00; A61K 47/02; A61K 9/70; A61K 9/0014; A61K 47/12

(57) ABSTRACT

A method of treating a topical ailments can include identifying an ailment, procuring a therapeutic composition, and administering the therapeutic composition to a subject. More specifically, the ailment can be associated with a topical region of a subject. The therapeutic composition can be sterilized and include a sodium carbonate mineral and a carboxylic acid. The therapeutic composition can be administered to the topical region of the subject.

### RELATED APPLICATION(S)

This application claims priority to U.S. Provisional Application No. 63/147,990, filed Feb. 10, 2021, which is incorporated herein by reference.

### BACKGROUND

Trona is a mineral found in various locations including California, Wyoming, Botswana, Egypt, and elsewhere. Trona can be formed by reactions of late magmatic fluids with earlier crystallized rocks or by vapor unmixing. The structure of trona includes three edge-sharing sodium polyhedral cross-linked by carbonate groups and hydrogen bonds. Trona has a wide range of uses including glass manufacturing, chemical manufacturing, paper manufacturing, and the like.

### SUMMARY

A method of treating a topical ailments can include identifying an ailment, procuring a therapeutic composition, and administering the therapeutic composition to a subject. More specifically, the ailment can be associated with a topical region of a subject. The therapeutic composition can be sterilized and include a sodium carbonate mineral and a carboxylic acid. The therapeutic composition can be administered to the topical region of the subject.

A trona based therapeutic device can include a trona based therapeutic composition contained in a structural carrier. The therapeutic composition is sterilized and includes a sodium carbonate mineral and a carboxylic acid.

A method of making a trona based therapeutic composition can include mixing a sodium carbonate mineral and a carboxylic acid to form a precursor mixture. The precursor mixture can be heated at a temperature and for a time sufficient to destroy substantially all bacteria and form a sterilized mixture. The sterilized mixture can be filtered to form a sterilized therapeutic composition.

There has thus been outlined, rather broadly, the more important features of the disclosure so that the detailed description thereof that follows may be better understood, and so that the present contribution to the art may be better appreciated. Other features of the present disclosure will become clearer from the following detailed description of the disclosure, taken with the accompanying drawings and claims, or may be learned by the practice of the disclosure.

### DETAILED DESCRIPTION

While these exemplary embodiments are described in sufficient detail to enable those skilled in the art to practice the disclosure, it should be understood that other embodiments may be realized and that various changes to the disclosure may be made without departing from the spirit and scope of the present disclosure. Thus, the following more detailed description of the embodiments of the present disclosure is not intended to limit the scope of the disclosure, as claimed, but is presented for purposes of illustration only and not limitation to describe the features and characteristics of the present disclosure, to set forth the best mode of operation of the disclosure, and to sufficiently enable one skilled in the art to practice the disclosure. Accordingly, the scope of the present disclosure is to be defined solely by the appended claims.

### Definitions

In describing and claiming the present disclosure, the following terminology will be used.

The singular forms “a,” “an,” and “the” include plural referents unless the context clearly dictates otherwise. Thus, for example, reference to “an acid” includes reference to one or more of such materials and reference to “heating” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%. Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well.

As used herein with respect to an identified property or circumstance, “substantially” refers to a degree of deviation that is sufficiently small so as to not measurably detract from the identified property or circumstance. The exact degree of deviation allowable may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

The term “coupled,” as used herein, is defined as directly or indirectly connected in a chemical, mechanical, electrical, or nonelectrical manner.

As used herein, a therapeutic composition is “sterilized” when the therapeutic composition passes a preservative efficacy test related to cosmetics products. In one aspect, the therapeutic composition passes a preservative efficacy test related to cosmetics products when the therapeutic composition passes a test including at least one of: a United States Pharmacopeia (USP)<61> Microbial Limits Test, a Personal Care Products Council (PCPC) M-1 “Determination of the Microbial Content of Cosmetic Products” test, a PCPC M-2 “Examination of Staphylococcus aureus, Escherichia coli and Pseudomonas aeruginosa” test, a PCPC M-3 “A Method for Preservation Testing of Water-Miscible Personal Care Products,” a PCPC M-4 “Method for Preservation Testing of Eye Area Cosmetics” test, a USP <2021>“Microbial Enumeration” test, a USP <2022>“Absence of Specified Microorganisms” test, an AOAC Official Method 991.14 “Coliform and Escherichia coli Counts in Foods” test, and a combination thereof. As used herein, Personal Care Products Council (PCPC) and Cosmetics, Toiletries, and Fragrance Association (CTFA) can be used to refer to the same entity or guideline.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate, modulate, or eliminate symptoms associated with a condition present in a subject, or can be prophylactic, (i.e., to prevent or reduce the occurrence of the symptoms in a subject). Such prophylactic treatment can also be referred to as prevention of the condition.

As used herein, the terms “therapeutic agent,” “active agent,” and the like can be used interchangeably and refer to agent that can have a beneficial or positive effect on a subject when administered to the subject in an appropriate or effective amount. In one aspect, the therapeutic or active agent can be a trona compound. The term “additional active agent” can be used to refer to a compound, molecule, or material other than a trona compound that has physiologic activity when administered to a subject in an effective amount.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format (e.g. a specific form, shape, vehicle, etc.) for administration to a subject. For example, a topical dosage form can be suitable for administration to a subject's skin by rubbing, etc.

As used herein, a “subject” refers to an animal. In one aspect the animal may be a mammal. In another aspect, the mammal may be a human.

As used herein, a “dosing regimen” or “regimen” such as an “initial dosing regimen” or “starting dose” or a “maintenance dosing regimen” refers to how, when, how much, and for how long a dose of the compositions of the present disclosure can be administered to a subject. For example, an initial or starting dose regimen for a subject may provide for a total topical daily dose of from about 1-5 sprays, squirts, pumps, etc. administered in two divided doses at least 12 hours apart (e.g., one at morning and one at night) for one week. As used herein, “daily dose” refers to the amount of active agent (e.g., trona) administered to a subject over a 24-hour period of time. The daily dose can be administered two or more administrations during the 24-hour period. In one embodiment, the daily dose provides for two administrations in a 24-hour period. With this in mind, an “initial dose” or initial daily dose” refers to a dose administered during the initial regimen or period of a dosing regimen.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein “single unit” when used to describe dosing of a subject refers to the dosage form being a single dosage form, e.g., a single tablet, capsule, pump or squirt of gel or solution, etc. In contrast, “multiple unit” when used to describe dosing of a subject refers to the dosage including two or more dosage forms, e.g. 2 tablets, 3 capsules, 2-4 pumps or squirts, etc. It is noteworthy that multiple unit dosage forms generally will be the same type of dosage forms (i.e., tablet or capsule) but may not be the same dosage form type.

As used herein, a “treatment situs” refers to a location on or within a subject where treatment is desired. For example, when treating a burn or a lesion, the treatment situs can be the area of the burn or lesion. Further, as used herein, an “application situs” refers to a location on or in a subject where treatment is administered. The application situs for a topical dosage formulation may be the area of skin or mucosa to which the topical dosage formulation is applied. In some embodiments, the application situs may be substantially the same as the treatment situs (e.g., the composition or formulation is administered directly to the treatment site). In other embodiments, the application situs may be different from (e.g., distal from) the treatment situs. In such cases, despite the fact that administration may be distal from the treatment situs, the composition or formulation still exerts a therapeutic effect at the treatment situs.

As used herein, “topical composition” or “topical administration” and the like refer to a composition suitable for administration directly to a skin or mucosa surface and from which an effective amount of a drug is released. In some embodiments, topical compositions can provide a local or localized therapeutic effect (e.g., at or near an application situs). For example, a topical composition when applied to a wound, a lesion, a burn, a canker sore, etc. (e.g., a treatment situs), may primarily exert a therapeutic effect at or around the application situs, but not substantially beyond it. In other embodiments, a topical composition can provide a regional effect. For example, a topical composition administered to a skin surface on a region of the body, such as a finger, arm, ankle, joint, etc. can exert a therapeutic effect within the region, but not substantially beyond. For example, a topical composition administered to the region of an ankle can have a therapeutic effect in and around the ankle, by for example, reducing edema, joint inflammation, pain, etc. In other embodiments, topical compositions can provide a systemic effect. In some aspects, a topical composition can provide the therapeutic effect though a mechanism of action where the drug or active agent itself arrives at the treatment situs. In other aspects, the topical composition can provide the therapeutic effect through an intermediate mechanism of action, such as biochemical cascade event, such as an enzymatic cascade or other signaling (e.g., cellular signaling, or inter/intra cellular signaling) event which ultimately exerts the desired therapeutic effect at the treatment situs. In some examples, such intermediate mechanism can allow treatment of a treatment situs that is distal from an application situs. In yet other examples, when treatment of a distal treatment situs occurs, the active agent may travel through dermal and other tissues from the application situs to the treatment situs and exert a direct effect.

As used herein, “co-administering” a first therapeutic agent with a second therapeutic agent can include concomitant administration within a suitable time window. In one example, the suitable time window can be less than one or more of: 1 hour, 45 minutes, 30 minutes, 15 minutes, 5 minutes, 2 minutes, 1 minute, or combinations thereof. Concomitant administration can be from the same composition or from different compositions.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, a burn, a sting, and the like. Some examples of chronic conditions can include without limitation, eczema, arthritis, and the like.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

In this disclosure, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the disclosure should be determined solely by the appended claims and their legal equivalents, rather than by the descriptions and examples given herein.

Occurrences of the phrase “in one embodiment,” or “in one aspect,” or in one example,” herein do not necessarily all refer to the same embodiment or aspect.

Specific examples of trona-based compositions and various other features or elements are shown in U.S. patent application Ser. No. 13/113,226, filed Mar. 23, 2011, and Ser. No. 13/841,902, filed Mar. 15, 2013, each of which is incorporated herein by reference in its entirety.

Trona Based Therapeutic Compositions and Use and Manufacture Thereof An initial overview of disclosure embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Trona (Na₂CO₃, NaHCO₃2H₂O) is a mineral that is mined in various locations. Trona has various uses but its use as a therapeutic agent has not been determined (e.g., as a topical formulation). When using a therapeutic agent as a topical formulation, the degree of sterilization should be tightly controlled to prevent the topical formulation from causing infections.

Various organizations are involved in setting standards for topical formulations. For example, the United States Pharmacopeia (USP) is a non-governmental, non-profit organization that has been setting standards for drugs for several years. Furthermore, the Personal Care Products Council (PCPC) is a leading trade organization representing cosmetics and personal care products. Therefore, complying with the standards and guidelines set by these organizations can be useful in formulating topical compositions.

In one embodiment, a method of treating a topical ailments can include identifying an ailment, procuring a therapeutic composition, and administering the therapeutic composition to a subject. More specifically, the ailment can be associated with a topical region of a subject. The therapeutic composition can be sterilized and can include a sodium carbonate mineral and a carboxylic acid. The therapeutic composition can be administered to the topical region of the subject.

In another embodiment, a trona based therapeutic device can comprise a trona based therapeutic composition contained in a structural carrier, wherein the therapeutic composition is sterilized and includes a sodium carbonate mineral and a carboxylic acid.

In yet another embodiment, a method of making a trona based therapeutic composition can comprise mixing a sodium carbonate mineral and a carboxylic acid to form a precursor mixture. In one aspect, the method can comprise heating the precursor mixture at a temperature and for a time sufficient to destroy substantially all bacteria and form a sterilized mixture. In another aspect, the method can comprise filtering the sterilized mixture to form a sterilized therapeutic composition.

### Compositions:

The therapeutic compositions can include the sodium carbonate mineral and the carboxylic acid. The sodium carbonate mineral can include trona, gaylussite, natron, prissonite, northupite, nahcolite, thermonatrite, and combinations of these minerals. Typically, the sodium carbonate mineral can be sterilized and separated from rock or other debris.

The carboxylic acid can be one or more mono-, di- or tricarboxylic acids. The carboxylic acids may be any of a wide variety of mono-, di- and tricarboxylic acids. These carboxylic acids may be comprised of mono-, di- or tricarboxylic acids having the general formula:

(HOOC)—R—(COOH)ₓ₋₁

where x is an integer of 1, 2 or 3, and R is a saturated or unsaturated, straight, or branched carbon chain having one to eighteen carbon atoms, or an aromatic moiety having six to eighteen carbon atoms which may be substituted or unsubstituted by OH, COOH, COOM, COOR′, —OR′ substituents, where M can be an alkali or alkaline earth metal, and where R′ can be saturated or unsaturated, straight, or branched carbon chain having from one to eight carbons, an aromatic moiety having six to eighteen carbon atoms which may be substituted by alkyl groups having one to eight carbons, OH, COOH, COOM, COOR′, —OR′ substituents, and M can be an alkali or alkaline earth metal. For purposes described herein salicylic acid and citric acid are particularly useful carboxylic acids with citric acid providing exceptional results, although other carboxylic acids can also be used. Suitable carboxylic acids can be used singly or in combination with multiple carboxylic acids. Representative, but not inclusive, of such carboxylic acids can include monocarboxylic acids such as acetic acid, propionic acid, butyric acid, valeric acid, caproic acid, caprylic acid, capric acid, lauric acid, myristic acid, palmitic acid, stearic acid, myristoleic acid, palmitoleic acid, sapienic acid, oleic acid, α-linolenic acid, o-phthalic acid, isophthalic acid, terephthalic acid, acetylsalicylic acid, and salicylic acid. Non-limiting examples of dicarboxylic saturated and unsaturated acids can include oxalic acid, malonic acid, succinic acid, glutaric acid, adipic acid, pimelic acid, suberic acid, azelaic acid, sebacic acid, maleic acid, fumaric acid, glutaconic acid, and traumatic acid. Similarly, non-limiting examples of tricarboxylic acids can include citric acid, isocitric acid, propane-1,2,3-tricarboxylic acid, aconitic acid, and trimesic acid.

In some embodiments, the composition can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof.

In one embodiment, the trona can be present in the composition at a concentration of from about 0.0001 wt % to about 10 wt %. In one example, the trona can be present in the composition at a concentration of from about 0.0001 wt % to about 5 wt %. In another example, the trona can be present in the composition at a concentration of from about 0.001 wt % to about 1 wt %. In one more example, the trona can be present in the composition at a concentration of from about 1.0 wt % to about 5.0 wt %.

The ratio of trona to carboxylic acid may vary on a weight basis (w/w). Although other ratios may be useful, as a general guideline, the ratio can often range from 200:1 to 5:1, and in some cases can be from 30:0.5 to 5:1. For example, in the case of citric acid, ratios of about 9:1 are particularly effective. The ratio can depend on the particular carboxylic acid, the number of acid groups and other functional groups, the molecular weight and other factors that can be determined systematically. In one case, the carboxylic acid is one or both of citric acid and salicylic acid. In another example, the carboxylic acid consists essentially of citric acid.

In one embodiment, the composition can further comprise an additional active agent selected from the group consisting of: an antioxidant, an anti-infective agent, an antibiotic, an anti-tumor agent, an anti-inflammatory agent, an analgesic, an anesthetic, an anti-rheumatic agent, a growth factor, a cytokine, an amino acid, a protein, a hormone, a vitamin, the like, and combinations thereof.

The additional active agent can be present at various amounts. In one aspect, the additional active can be present at a concentration of from about 0.0001 wt % to about 10 wt %. In another aspect, the additional active can be present at a concentration of from about 0.0001 wt % to about 1 wt %. In one more aspect, the additional active can be present at a concentration of from about 0.001 wt % to about 1 wt %. In yet one more aspect, the additional active can be present at a concentration of from about 0.1 wt % to about 1 wt %.

Antioxidants can be included in the present compositions and dosage forms for their effect as an additional active agent. Various antioxidants can be used including, but not limited to: N-acetyl-cysteine, hydroxytyrosol (HXT), reduced glutathione (rGSH), catalase, Vitamin A, Vitamin C, Vitamin E, coenzyme Q10, managanese, iodide, melatonin, alpha-carotene, astaxanthin, beta-carotene, canthaxanthin, cryptoxanthin, lutein, lycopene, zeaxanthin, apigenin, luteolin, tangeritin, isorhamnetin, kaempferol, myricetin, proanthocyanidins, quercetin, eriodyctiol, hesperetin, naringenin, catechin, gallocatechin, epicatechin, epigallocatechin, theaflavin, thearubigins, daidzein, genistein, glycitein, resveratrol, pterostilbene, cyanidin, delphinidin, malvidin, pelargonidin, peonidin, petunidin, chicoric acid, cholorogenic acid, cinnamic acid, ellagic acid, ellagitannins, gallic acid, gallotannins, rosmarinic acid, slaicyclic acid, curcumin, flavonolignans, xanthones, eugenol, capsaicin, bilirubin, citric acid, oxalic acid, phytic acid, R-alpha-Lipoic acid, the like, and combinations thereof.

The additional active agent can include one or more anti-infective agents, namely an agent that can kill or prevent an infectious organism (e.g., a pathogen) from spreading. Thus, anti-infective agents can include antibacterial agents, antifungal agents, antiviral agents, antiprotozoan agents, the like, or combinations thereof. Non-limiting examples can include amebicides such as chloroquine, nitazoxanide, paromomycin, tinidazole, metronidazole, iodoquinole, or the like; aminoglycosides such as tobramycin, gentamicin, amikacin, kanamycin, neomycin, streptomycin, or the like; anthelmintics such as albendazole, ivermectin, praziquantel, pyrantel, mebendazole, miltefosine, niclosamide, piperazine, thiabendazole, or the like; antifungals such as itraconazole, posaconazole, ketoconazole, fluconazole, clotrimazole, isavuconazole, miconazole, voriconazole, echinocandins, terbinafine, griseofulvin, flucytosine, nystatin, amphotericin b, or the like; antimalarials such as chloroquine, quinine, hydroxychloroquine, mefloquine, primaquine, pyrimethamine, halofantrine, doxycycline, or the like; antituberculosis agents such as aminosalicylic acid, bedaquiline, isoniazid, ethambutol, pyrazinamide, ethionamide, rifampin, rifabutin, rifapentine, capreomycin, cycloserine, streptomycin, or the like; antivirals such as amantadine, rimantadine, ritonavir, cobicistat, peginterferon alfa-2a, peginterferon alfa 2b, maraviroc, raltegravir, dolutegravir, elvitegravir, sofosbuvir, enfuvirtide, fomivirsen, foscarnet, oseltamivir, zanamivir, peramivir, etravirine, efavirenz, nevirapine, delavirdine, rilpivirine, daclatasvir, adefovir, entecavir, telbivudine, didanosine, tenofovir, abacavir, lamivudine, zidovudine, stavudine, emtricitabine, zalcitabine, boceprevir, simeprevir, fosamprenavir, lopinavir, darunavir, telaprevir, ritonavir, tipranavir, atazanavir, nelfinavir, amprenavir, indinavir, saquinavir, ganciclovir, valacyclovir, famciclovir, acyclovir, valganciclovir, ribavirin, cidofovir, or the like; carbapenems such as doripenem, meropenem, cilastatin, ertapenem, or the like; cephalosporins such as avibactam, ceftolozane, ceftazidime, tazobactam, cefadroxil, cephalexin, cefazolin, ceftaroline, loracarbef, cefotetan, cefuroxime, cefprozil, cefaclor, cefoxitin, ceftibuten, cefotaxime, ceftriaxone, cefpodoxime, cefixime, cefdinir, defditoren, ceftazidime, ceftizoxime, cefepime, or the like; glycopeptide antibiotics such as vancomycin, dalbavancin, oritavancin, telavancin, or the like; glycocyclines such as tigecycline, or the like; leprostatics such as thalidomide, dapsone, clofazimine, or the like; lincomycin, or the like; clindamycin, or the like; ketolides such as telithromycin, or the like; macrolides such as azithromycin, fidaxomicin, erythromycin, clarithromycin, or the like; antibiotics such as aztreonam, daptomycin, chloramphenicol, colistimethate, fosfomycin, rifaximin, metronidazole, sulfamethoxazole, atovaquone, bacitracin, dalfopristin, erythromycin, furazolidone, pentamidine, polymyxin b, spectinomycin, trimetrexate, linezolid, tedizolid, penicillins (e.g. ampicillin, amoxicillin, carbenicillin, piperacillin, ticarcillin, nafcillin, dicloxacillin, cloxacillin, oxacillin, or the like), quinolones (e.g. lomefloxacin, norfloxacin, ofloxacin, gatifloxacin, moxifloxacin, ciprofloxacin, levofloxacin, gemifloxacin, cinoxacin, nalidixic acid, sparfloxacin, or the like), sulfonamides (e.g. sulfamethoxazole, sulfadiazine, sulfisoxazole, or the like), tetracyclines (e.g. tetracycline, demeclocycline, doxycycline, minocycline, or the like), or the like; urinary anti-infectives such as methenamine, methylene blue, fosfomycin, nitrofurantoin, trimethoprim, cinoxacin, nalidixic acid, oxytetracycline, or the like; hydrates thereof, acids thereof, bases thereof, salts thereof, or combinations of any of such anti-infective agents.

In some examples, the additional active agent can also include any suitable antitumor agent. Non-limiting examples of antitumor agents can include angiogenesis inhibitors such as angiostatin k1-3, angiostatin k1-5, DL-α-difluoromethylornithine, endostatin, fumagillin, genistein, minocycline, staurosporine, (+/−)-thalidomide, or the like; DNA intercalators such as bleomycin, carboplatin, carmustine, chlorambucil, cisplatin, cyclophosphamide, cis-diammineplatinum(II) dichloride, melphalan, mitoxantrone, oxaliplatin, or the like; DNA synthesis inhibitors such as (+/−)-amethopterin, 3-amino-1,2,4-benzotraizine-1,4-dioxide, aminopterin, cytosine β-D-arabinofuranoside, 5-fluoro-5′-deoxyuridine, 5-fluorouracil, ganciclovir, hydroxyurea, mitomycin C, or the like; transcriptioin regulators such as actinomycin D, daunorubicin, doxorubicin, homoharringtonine, idarubicin, or the like; enzyme inhibitors such as S(+)-camptothecin, curcumin, (−)-deguelin, 5,6-dichlorobenzimidazole 1-β-D-ribofuranoside, etoposide, formestane, fostriecin, hispidin, 2-imino-1-imidazolidineacetic acid, mevinolin, trichostatin A, tyrphostin AG 34, tyrphostin AG 879, or the like; gene regulation agents such as 5-aza-2′-deoxycytidine, 5-azacytidine, cholecalciferol, 4-hydroxytamoxifen, melatonin, mifepristone, raloxifene, vitamin A aldehyde, vitamin A acid, vitamin A, 9-cis-retinoic acid, 13-cis-retinoic acid, tamoxifen, troglitazone, or the like; microtubule inhibitors such as colchicine, docetaxel, dolastatin 15, etoposide, irinotecan, nocodazole, paclitaxel, podophyllotoxin, rhizoxin, vinblastine, vincristine, vindesine, vinorelbine, or the like; other antitumor agents such as 17-(allylamino)-17-demethoxygeldanamycin, 4-amino-1,8-naphthalimide, apigenin, brefeldin A, cimetidine, dichloromethylene-diphosphonic acid, leuprolide, luteinizing hormone-releasing hormone, pifithrin-α, rapamycin, sex hormone-binding globulin, thapsigargin, bikunin, ifosfamide, temozolomide, capecitabine, methotrexate, gemcitabine, pemetrexed, mitomycin, epirubicin, bevacizumab, cetuximab, gefitinib, imatinib, trastuzamab, denosumab, rituximab, sunitinib, zoledronate, abiraterone, anastrozole, bicalutamide, exemestane, goserelin, medroxyprogesterone, octreotide, tamoxifen, bendamustine, lomustine, procarbazine, streptozocin, fludarabine, raltitrexed, mitoxantrone, eribulin, topotecan, afatinib, aflibercept, BCG, crizotinib, dabrafenib, interferon, ipilimumab, lapatinib, nivolumab, panitumumab, pembrolizumab, pertuzumab, sorafenib, trastuzumab emtansine, temsirolimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, or the like; hydrates thereof, acids thereof, bases thereof, salts thereof, or combinations of any of such antitumor agents.

In some examples, the additional active agent can also include any suitable anti-inflammatory agent. Non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, durasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof, or combinations thereof.

### Manufacturing and Sterilization:

A method of making the therapeutic compositions can include mixing a sodium carbonate mineral and a carboxylic acid to form a precursor mixture. The precursor mixture can be heated at a temperature and for a time sufficient to destroy substantially all bacteria and form a sterilized mixture. The sterilized mixture can also be filtered to form a sterilized therapeutic composition. The heating time can vary but is often from 7 to 20 minutes. Furthermore, the temperature can be above a boiling point of the precursor mixture.

In one example, a mixture of trona and citric acid at a 10:1 weight ratio is formed. This mixture can then be dissolved in distilled water. Weight ratios of powder to water can range from about 1:1 to 1:30 depending on the desired concentration, and in some cases 1:10 to 1:15. The dissolved solution can then be heated to boiling and held for a desired heating time. Natural debris and precipitated solids can then be removed through simple mechanical filtering to produce a sterilized therapeutic composition.

In one example, the therapeutic composition can be sterilized when the therapeutic composition passes a preservative efficacy test comprising at least one of: a United States Pharmacopeia (USP)<61> Microbial Limits Test, a Personal Care Products Council (PCPC) M-1 “Determination of the Microbial Content of Cosmetic Products” test, a PCPC M-2 “Examination of Staphylococcus aureus, Escherichia coli and Pseudomonas aeruginosa” test, a PCPC M-3 “A Method for Preservation Testing of Water-Miscible Personal Care Products,” a PCPC M-4 “Method for Preservation Testing of Eye Area Cosmetics” test, a USP <2021>“Microbial Enumeration” test, a USP <2022>“Absence of Specified Microorganisms” test, an AOAC Official Method 991.14 “Coliform and Escherichia coli Counts in Foods” test, and a combination thereof.

In one aspect, the therapeutic composition can be sterilized when the therapeutic composition passes a preservative efficacy test (a) after 2 weeks when stored at room temperature and 50-75% relative humidity; or (b) after 4 weeks when stored at room temperature and 75% relative humidity, or (c) after 2 weeks when stored at 40° C. and 75% relative humidity, or (d) after 4 weeks when stored at 40° C. and 75% relative humidity.

In yet another embodiment, a trona-based composition can include a container configured to minimize an amount of pathogens in the container. In one example, the container as used herein can be a container that is made of stainless steel or other suitable non-reactive material. Such containers may include glass-lined containers, polymeric bags, or other suitable containers that are generally employed in the compounding of pharmaceutical liquid preparations. In another example, the container can be a glass or plastic vial which contains the composition. Depending on the sterility restrictions, these vials may be further sterilized using heat, steam, gas, or radiation, among others.

### Formulations and Dosage Forms

During administration, the dosage can vary depending on the particular ailment, severity of conditions, age of patient, body mass of patient, and the like. Regardless, administering can include selecting a dosage sufficient to produce a therapeutic effect on the ailment and contacting the topical region with the therapeutic composition.

As an effective delivery vehicle, a trona based therapeutic device can include the therapeutic composition contained in a structural carrier where the therapeutic composition is sterilized and includes a sodium carbonate mineral and a carboxylic acid. The structural carrier can be a fabric wipe, a compress, a bandage, or the like.

Depending on the intended carrier and mode of application, the sterilized therapeutic composition can be introduced onto the appropriate substrate. Furthermore, the therapeutic composition can be prepared as a cream, lotion, gel, or the like.

The topical composition can have any suitable pH for topical application. In one example, the topical composition can have a pH of from about 6.8 to about 7.4. In another example, the topical composition can have a pH of from about 7.0 to about 7.2.

The topical composition can have any suitable viscosity for applying a topical substance. In one example, the topical composition can have a viscosity of from about 1 cP to about 15 cP. In another example, the topical composition can have a viscosity of from about 20 cP to about 100 cP. In one more example, the topical composition can have a viscosity of from about 300 cP to about 1000 cP.

For example, carriers or bases may be added to the composition such as water, glycerin, ethanol, propylene glycol, isopropyl alcohol, vegetable oils such as soybean, avocado, sunflower, sesame seed oil, peanut oil, or other nut oils, canola, almond, jojoba oil, olive oil, safflower oil, grape seed oil, cranberry seed oil, sea buckthorn oil, any fatty acid ester such as isopropyl myristate, capryllic alcohol and esters.

In addition one or more homeopathic agents or ingredients can be added such as acidifying agents, alkalizing agents, antimicrobial agents, antioxidants, buffering agents, chelating agents, coloring additives, dispersing agents, emollients, emulsifying agents, humectants, fragrances, masking agents, preservatives, sugars, sunscreen agents, surfactants, suspending agents, or thickening agents.

Acidifying and alkalizing agents may be added to obtain the desired pH of the therapeutic composition. The acidifying agents may be at least one of acetic acid, citric acid, glacial acetic acid, malic acid, or proprionic acid. The alkalizing agents may be edetates, potassium carbonate, potassium hydroxide, sodium borate, sodium carbonate, or sodium hydroxide.

Anti-microbial agents may be used when the skin area to be treated may be prone to microbial infection, bacteria, fungal, or protozoa. Anti-microbial agents may be benzyl alcohol, potassium sorbate, sorbic acid, benzoic acid, butyl paraben, ethyl paraben, methyl paraben, propyl pareben, or sodium benzoate.

Antioxidants may be used to protect ingredients from oxidizing agents that are included within or come in contact with the therapeutic composition. The antioxidants may be at least one of water-soluble antioxidants such as all poly phenols, propyl gallate, ascorbic acid, carnosine, N-acetyl-carnosine, and cysteine hydrochloride and oil-soluble antioxidants such as retinoids, retinoic acid, all carotenes, ascorbyl palmitate, bulytlated hydroxyanisole, butylated hydroxytoluene, octyl gallate, dodecyl gallate, phenyl-alpha-napthyl-amine, tocotrienols, and tocopherols such as alpha-tocopherol. Buffering agents may be used to maintain an established pH. The buffering agents may be at least one of sodium citrate, calcium acetate, potassium metaphosphate, potassium phosphate monobasic, or tartaric acid.

Chelating agents may also be used to maintain an ionic strength of the composition and/or bind to destructive compounds and metals that may be included within or come in contact with the composition. Non-limiting examples, of suitable chelating agents can be at least one of dihydroxy ethyl glycine, amino acids, citric acid, tartaric acid, edetate dipotassium, edetate disodium, edetic acid, or ethylenediamine tetracetic acid (EDTA) and its salts (e.g., tetrasodium EDTA).

Coloring additives may be used to add aesthetic color to the therapeutic composition. Coloring additives may be, but are not limited to, titanium dioxide, yellow iron oxide, red iron oxide, black iron oxide, caramel, carmine, fluorescein derivatives, methoxsalen, trioxsalen, carbon black, azo dyes, anthraquinone dyes, blue azulenes, guajazulene, chamuzulene, erythrosin, bengal rose, phloxin, cyanosin, daphinin, eosin G, cosin 108, or Acid Red 51 or any of the FD&C approved colors designated for food and cosmetic use.

Dispersing and suspending agents may be quarternium-18 hectorite, polyhydroxy stearic acid, magnesium stearate, or other fatty acid salts, poligeenan, or silicon dioxide.

Emollient agents may soften and smooth the skin. The emollients may be at least one of hydrocarbon such as mineral oil, petrolatum, microcrystaline wax, polyethylene, triglyceride esters such as those of castor oil, cocoa butter, safflower oil, cottonseed oil, corn oil, olive oil, cod liver oil, almond oil, avocado oil, palm oil, sesame oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholipids, or sterols.

Emulsifying agents may be used when the composition is formed as an emulsion. The emulsifying agents used for preparing water-in-oil emulsions may be cyclomethicone (and) dimethicone copolyol, dimethicone copolyol, cetyl dimethicone copolyol, PEG-30 dipolyhydroxystearate, or PEG-40 sorbitan peroleate. The emulsifying agents used for preparing oil-in-water emulsions may be glyceryl stearate, PEG-100 stearate, methyl gluceth sesquisterate, fatty alcohols, or alkyl phenols condensed with ethylene oxide.

Humectants may promote the retention of moisture, e.g., moisturizers. The humectants may be at least one of sorbitol, matricaria extract, aloe barbadensis gel, glycerin, glycereth 5 lactate, glycereth 7 triacetate, glycereth 7 diisononoate, hexanetriol, hexylene glycol, propylene glycol, dipropylene glycol, alkoxylated glucose, D-panthenol, 1-2-pantandiol, 2-methyl-1,3-propanediol, and derivatives thereof, or hyaluronic acid.

Fragrances may be at least one of peppermint, rose oil, rose water, aloe vera, clove oil, menthol, camphor, or eucalyptus oil. Certain fragrances use a solubilizer, e.g., PPG-5-ceteareth-20. To eliminate certain odors from compositions, masking agents may be used. An example of a masking agent may be ethylene brassylate.

Preservatives may also be used to protect the therapeutic composition from degradation. Non-limiting examples of preservatives can include phenoxyethanol, methyl paraben, propyl paraben, butyl paraben, isopropyl paraben, isobutyl paraben, dieizolidinyl urea, imidazolidinyl urea, diazolindyl urea, benzalkonium chloride, benzethonium chloride, or phenol.

Sunscreen agents may also be added to the therapeutic composition to block or reduce the amount of ultraviolet radiation impinging on the skin and to augment blocking effects of the mineral and carboxylic acid components. The sunscreen agents may be organic compounds and/or inorganic materials. Non-limiting examples of the organic compounds can include at least one of oetyl methoxycinnamate, oetyl salicylate, benzophenone-3 homosalate, octocrylate, avobenzone, or menthyl anthranilate. The inorganic materials may be at least one of zinc oxide, silica, iron oxide, titanium dioxide, or 2-ethyl-hexyl-p-methoxycinnamate.

Surfactants may be used to stabilize the therapeutic compositions and may be used as wetting agents, antifoam agents, emulsifiers, dispersing agents, or penetrant agents. Suitable surfactants may include at least one of methyl gluceth 20, decyl polyglucoside, lapyrium chloride, laureth 4, laureth 9, monoethanolamine, nonoxynol 4, nonoxynol 9, nonoxynol 10, nonoxynol 15, nonoxynol 30, poloxalene, polyoxyl 8, 40, and 50 stearate, polysorbate 20, polysorbate 40, polysorbate 60, polysorbate 65, polysorbate 80, polysorbate 85, sodium lauryl sulfate, or sorbitan and its derivatives.

Similarly, at least one humectant may be included when the therapeutic composition is an aqueous composition. The humectant may reduce the tackiness produced by the aqueous composition after it has been applied and has dried on the skin. The humectants may be present in an amount such as to produce an aqueous composition that is capable of being substantially non-tacky after drying on the skin. The humectants may be at least one of glycerin, cellulose, or poly saccharides.

In one example, the dosage form can have from about 0.1 ml to about 10 ml of the composition. In another example, the dosage form can have from about 0.5 ml to about 1 ml of the composition. In another example, a dosage form can be from about 10 ml to about 20 ml of the composition. In another example, a daily dose can be from about 0.1 ml to about 100 ml of the composition.

In one more example, the dosage interval can vary based on the number of previous doses administered in a selected time window and a duration of administration. In one example, the therapeutically effective amount of the topical composition can be administered to a subject at least two different dosage intervals. The first dosage interval (e.g., the time between the first dose administered and the second dose administered in a day) can be a selected interval between 30 minute and 12 hours and the second dosage interval (e.g., the time between the second dose administered and the third dose administered in a day) can be a selected interval between 30 minutes and 12 hours. In some examples, the first dosage interval can be lower than a second dosage interval. For example, a subject who receives a first dose at 9 am, a second dose at 12 pm, and a third dose at 10 pm would have a first dosage interval of about 3 hours and a second dosage interval of about 10 hours. In this example, the dosage interval can lengthen over the course of a day. In other example, the dosage interval can lengthen over the course of a week, 2 weeks, 4 weeks, 8 weeks, 3 months, 6 months, 1 years, the like, and combinations thereof. In one example, a second topical composition can be administered within about 15 seconds, 20 seconds, 25 seconds, 30 seconds, 35 seconds, 45 seconds, 60 seconds, 5 minutes, 10 minutes, or 15 minutes seconds of the first administration.

### Methods of Treatment:

The ailments which can be effectively treated can include burns, skin irritations, wounds, skin allergens, and neuropathy care. For example, the ailment can be one of more of fire burns, sunburns, abrasions, wounds, medical burns, insect bite, insect sting, skin cut, cold sore, itch, rash, poisonous plant exposure, mouth irritation, warts, corns, razor abrasions, athletes foot, nail fungus, cuticle infection, dry skin, eczema, psoriasis, scalp irritation, sores, fever, peripheral neuropathy, neuropathy, edema, erythema, pain, scarring, venous insufficiency disease, muscle soreness, and disinfection.

Non-limiting examples of ailments can include application to 1^{st}, 2^{nd} and 3^{rd} degree burns while providing pain relief and helps healing, abrasions. Sunburns can also be treated with a reduction of heat from skin, while also removing redness and facilitating healing. Medical burns such as those resulting from laser, chemical, chemotherapy, welders or other exposures can also be treated.

These therapeutic compositions can also be used prophylactically. For example, the compositions can be used as sunscreen to help prevent sunburns, e.g. SPF 30 and above. Insect bite pain relief and insect sting pain relief can also be treated including pain and itch relief, speeds healing, and shrinks swelling. These compositions can also treat topical skin cuts and provide pain relief and increased rates of healing.

These compositions can also provide cold sore and canker sore relief and speeds heals, itch relief from poisonous plants and bug bites, as well as itching from recent transplant patients while avoiding interaction with drugs already being taken by patients.

Other treatable ailments can include mouth irritations, healing of fungus, trench mouth and gum disease, wart and corns, razor skin irritations associated with shaving—facial, leg, arms etc, athletes foot, toe nail fungus, cuticle infections, dry skin (heals and moisturizes), diabetes and liver patients (helps relieve itching from buildup of phosphorus and gas), eczema and psoriasis, UV damage to premature infants, scalp irritations and sores, bed sores (helps healing and promotes skin rebuilding), diabetic sores (helps heal and promotes skin rebuilding), neuropathy (relieve the pain in their extremities), peripheral neuropathy, fever (lowers temperature, helps break the fever, and removes heat from the surface of the skin), edema (swelling), and the like. Other treatable ailments include rashes caused by poisonous plants exposure, muscle soreness, erythema, pain, scarring, venous insufficiency disease, and the like.

As a specific example, fevers can be effectively and sometimes dramatically reduced through topical application of the therapeutic compositions. A fever is a temporary increase in your body temperature, often due to an illness. For an adult, a fever may be uncomfortable, but usually isn't a cause for concern unless it reaches 103 F (39.4 C) or higher. The patient can have nonspecific symptoms such as mild headache, fatigue, general malaise, and fleeting aches and pains. Second stage or chill. The patient will feel chilled and develop generalized shaking despite his rising temperature. Vasoconstriction and piloerection precede the onset of shivering. Another name for a fever is pyrexia. Febrile is another term for fever. Afebrile means the absence of fever (body temperature within normal limits WNL). Fever occurs because our body is having a degree of inflammation or irritation, which causes the cells within the body to bump into each other quickly, which causes an increase in body heat. It is how rapidly the fever occurs that is more of a concern than the degree of the fever (unless it reaches dangerous levels).

These therapeutic compositions can remove heat from the skin. Prior testing has shown removal of pain from a burn, i.e. sunburn, burn from a hot stove, etc. Thus, if liquid therapeutic composition is placed on a pad, similar to a packaged towelette, it can be placed on a forehead or neck to aid in breaking the heat from a fever. In one example, the liquid therapeutic composition can be impregnated on a towelette. Such impregnated devices can be individually packaged for portability. Such devices can be used in emergency rooms, hospitals, or as a consumer retail product in stores to be used to break a fever. Thus, eliminating medication such as Tylenol, etc which when taken internally can be overused and become toxic. These therapeutic compositions can also be used to reduce blood spoilage by helping to prolong storage of blood. In another alternative, these therapeutic compositions can be used contemporaneously with other active agents to enhance performance of other drugs.

Administering the therapeutically effective amount of the topical composition can reduce the symptoms associated with the condition being treated. In one aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In still another alternative, these therapeutic compositions can be used as a hand sanitizer or surface disinfectant. In one aspect, when an effective amount of the therapeutic composition is applied to treatment situs, a selected amount of bacteria can be eliminated within a selected time. In one aspect, the selected amount of bacteria can be greater than at least one of 90%, 95%, 99%, 99.9%, the like or a combination thereof. In one aspect, the selected time can be less than at least one of: 5 minutes, 1 minute, 30 seconds, 15 seconds, 5 seconds, the like, or a combination thereof.

### EXPERIMENTAL EXAMPLES

The following examples are provided to promote a clearer understanding of certain embodiments of the present disclosure and are in no way meant as a limitation thereon.

### Example 1—Trona-Based Composition

A concentrated trona based composition was mixed in a 10:1 ratio of trona (e.g., 10 parts) and citric acid (e.g., 1 part). The concentrated trona based composition was mixed in a 1:10 ratio with distilled water to form a trona based composition. The trona based composition was mixed until dissolved. The concentrated trona based composition and the trona based composition are shown in Table 1-A and Table 1-B.

TABLE 1-A
Concentrated Trona based composition

| Ingredient | Percent (w/v) |
|---|---|
| Trona | 90.9% |
| Citric acid | 9.1% |

TABLE 1-B
Trona based composition

| Ingredient | Percent (w/v) |
|---|---|
| Trona | 8.3% |
| Citric acid | 0.8% |
| Distilled water | 90.9% |

### Example 2—Trona-Based Composition

A concentrated trona based composition was mixed in a 9:1 ratio of trona (e.g., 9 parts) and citric acid (e.g., 1 part). The concentrated trona based composition was mixed in a 1:9 ratio with distilled water to form a trona based composition. The trona based composition was mixed until dissolved and then strained. The concentrated trona based composition and the trona based composition are shown in Table 2-A and Table 2-B.

TABLE 2-A
Concentrated Trona based composition

| Ingredient | Percent (w/v) |
|---|---|
| Trona | 90% |
| Citric acid | 10% |

TABLE 2-B
Trona based composition

| Ingredient | Percent (w/v) |
|---|---|
| Trona | 9% |
| Citric acid | 1% |
| Distilled water | 90% |

### Example 3—Sterilized Trona-Based Composition

A concentrated trona based composition was mixed in a 9:1 ratio of trona (e.g., 9 parts) and citric acid (e.g., 1 part). The concentrated trona based composition was mixed in a 1:9 ratio with distilled water to form a trona based composition. The trona based composition was mixed until dissolved and then strained. The trona based composition was boiled for 7 minutes. The concentrated trona based composition and the trona based composition are shown in Table 3-A and Table 3-B.

TABLE 3-A
Concentrated Trona based composition

| Ingredient | Percent (w/v) |
|---|---|
| Trona | 90% |
| Citric acid | 10% |

TABLE 3-B
Trona based composition

| Ingredient | Percent (w/v) |
|---|---|
| Trona | 9% |
| Citric acid | 1% |
| Distilled water | 90% |

### Example 4—Additional Concentrated Trona-Based Compositions

A concentrated trona based composition can be mixed in various ratios of trona (e.g., 4, 9, 19, 49, 99, 199, parts) and citric acid (e.g., 1 part) as shown in Table 4-A.

TABLE 4-A
Concentrated Trona based composition

| Ingredient | 4a | 4b | 4c | 4d | 4e | 4f |
|---|---|---|---|---|---|---|
| Trona | 80% | 90% | 95% | 98% | 99% | 99.5% |
| Citric acid | 20% | 10% | 5% | 2% | 1% | 0.5% |

*All examples in w/v.

### Example 5—Additional Trona-Based Compositions

A trona based composition can be mixed in various ratios of trona (e.g., 4, 9, 19, 49, 99, 199, parts), citric acid (e.g., 1 part), and water (10 parts) as shown in Table 5-A.

TABLE 5-A
Concentrated Trona based composition

| Ingredient | 4a | 4b | 4c | 4d | 4e | 4f |
|---|---|---|---|---|---|---|
| Trona | 8% | 9% | 9.5% | 9.8% | 9.9% | 9.95% |
| Citric acid | 2% | 1% | 0.5% | 0.2% | 0.1% | 0.05% |
| Distilled | 90% | 90% | 90% | 90% | 90% | 90% |
| Water | | | | | | |

*All examples in w/v.

### Example 6—Preservative Efficacy Testing

### Method:

A concentrated trona-based sample of the trona-based composition consisting of a 10:1 ratio of trona (e.g., 10 parts) and citric acid (e.g., 1 part) was mixed. This concentrated sample of the trona-based composition was mixed with distilled water in a 1:10 ratio—e.g., 1 part of the concentrated sample (10 parts trona, 1 part citric acid) and 10 parts distilled water—to form a trona-based sample. This trona-based sample was strained and subject to testing against five individual microorganisms provided in Table 6 in accordance with the USP <61> method. Pure culture tests yielded specific data on each microorganism employed in the study.

### Results:

TABLE 6
Testing of Trona-Based Sample

| Microorganism | Percent Recovery | Pass/Fail |
|---|---|---|
| S. aureus | >200% | Fail |
| P. aeruginosa | >200% | Fail |
| E. coli | >200% | Fail |
| A. niger | 3% | Fail |
| C. albicans | 2% | Fail |

The test results show that this trona-based sample failed each of the tests against each individual microorganism.

### Example 7—Preservative Efficacy Testing

### Method:

A concentrated trona-based sample of the trona-based composition consisting of a 10:1 ratio of trona (e.g., 10 parts) and citric acid (e.g., 1 part) was mixed. This concentrated sample of the trona-based composition was mixed with distilled water in a 1:10 ratio—e.g., 1 part of the concentrated sample (10 parts trona, 1 part citric acid) and 10 parts distilled water—to form a trona-based sample. This trona-based sample was boiled for 3 minutes and then strained and subject to testing against five individual microorganisms provided in Table 7 in accordance with the USP <61> method. Pure culture tests yielded specific data on each microorganism employed in the study.

### Results:

TABLE 7
Testing of Trona-Based Sample

| Microorganism | Percent Recovery | Pass/Fail |
|---|---|---|
| S. aureus | 117% | Pass |
| P. aeruginosa | 50% | Pass |
| E. coli | 77% | Pass |
| A. niger | 80% | Pass |
| C. albicans | 90% | Pass |

The test results show that this trona-based sample passed each of the tests against each individual microorganism.

### Example 8—Preservative Efficacy Testing

### Method:

A sterile sample of the trona-based composition (Example 1-B) was tested against seven individual microorganisms provided in Table 8-A in accordance with CTFA M-3/M-4 guidelines. Pure culture tests yielded specific data on each microorganism employed in the study.

TABLE 8-A
Seven Individual Microorganisms

| Test Organisms | Inoculum Level (CFU/g) | ATCC Number |
|---|---|---|
| Escherichia coli | 1 × 10⁶ | 25922 |
| Klebsiella pneumoniae | 1 × 10⁶ | 700603 |
| Pseudomonas aeruginosa | 1 × 10⁶ | 9027 |
| Acinetobacter baumannii | 1 × 10⁶ | 19606 |
| Staphylococcus aureus | 1 × 10⁶ | 6538 |
| Candida albicans | 1 × 10⁵ | 10231 |
| Aspergillus niger | 1 × 10⁵ | 16404 |

The sample was initially tested for aerobic bacteria, yeast, and mold following the CTFA M-1/M-2 guidelines. This initial screen was used to ensure that the product does not contain any microorganisms prior to beginning the inoculations. No organisms were found in the sample during the initial screen.

### Effectiveness Standards:

99.9% reduction of bacteria within seven days.
90% reduction of yeasts and molds within seven days.
No increase for the duration of the test period.
Upon reinoculation, the same effectiveness standards apply.

### Conclusion:

The sample passed the test. The data collected from the study at each testing interval is shown in Table 8-B.

TABLE 8-B
Pure Culture Testing

| Interval | | E. coli | K. pneumoniae | P. aeruginosa | A. baumannii | S. aureus | C. albicans | A. brasiliensis |
|---|---|---|---|---|---|---|---|---|
| 0 | Hours | 21,000 | 710,000 | <10 | 940,000 | 1,300,000 | >250,000 | <10 |
| 24 | Hours | <10 | 730 | <10 | <10 | 450,000 | 2,800,000 | <10 |
| 48 | Hours | <10 | <10 | <10 | <10 | 130,000 | 200,000 | <10 |
| 72 | Hours | <10 | <10 | <10 | <10 | 23,000 | 120,000 | <10 |
| 1 | Week | <10 | <10 | <10 | <10 | 120 | 46,000 | <10 |
| 2 | Weeks | <10 | <10 | <10 | <10 | <10 | 2,300 | <10 |
| 3 | Weeks | <10 | <10 | <10 | <10 | <10 | 30,000 | <10 |
| 4 | Weeks | <10 | <10 | <10 | <10 | <10 | <10 | <10 |

*All results in Table 8-B are reported as CFU/g.

### Example 9—Preservative Efficacy Testing

### Method:

A concentrated trona-based sample of the trona-based composition consisting of a 10:1 ratio of trona (e.g., 10 parts) and citric acid (e.g., 1 part) was mixed. This concentrated sample of the trona-based composition was mixed with distilled water in a 1:10 ratio—e.g., 1 part of the concentrated sample (10 parts trona, 1 part citric acid) and 10 parts distilled water—to form a trona-based sample. This trona-based sample was boiled for 3 minutes and then strained. The sample was tested two weeks later, and subject to testing against five individual microorganisms provided in Table 9 in accordance with either the CTFA M-1 or CTFA M-2 guidelines. Pure culture tests yielded specific data on each microorganism employed in the study.

### Results:

TABLE 9
Testing of Trona-Based Sample After Two Weeks

| Microorganism | Amount/Concentration | Method |
|---|---|---|
| Total Aerobic | <10 (CFU/g) | CTFA M-1 |
| Microbial Count | | |
| E. coli | Absent | CTFA M-2 |
| | (per 10 grams) | |
| S. Aureus | Absent | CTFA M-2 |
| | (per 10 grams) | |
| Salmonella | Absent | CTFA M-2 |
| | (per 10 grams) | |
| Total Yeast and | <10 (CFU/g) | CTFA M-1 |
| Mold Count | | |
| Yeast | <10 (CFU/g) | CTFA M-1 |
| Mold | <10 (CFU/g) | CTFA M-1 |
| P. Aeruginosa | Absent | CTFA M-2 |
| | (per 10 grams) | |

The test results show that this trona-based sample prevented the growth of microorganisms over the course of 14 days.

### Example 10—Preservative Efficacy Testing

### Method:

A concentrated trona-based sample of the trona-based composition consisting of a 10:1 ratio of trona (e.g., 10 parts) and citric acid (e.g., 1 part) was mixed. This concentrated sample of the trona-based composition was mixed with distilled water in a 1:10 ratio—e.g., 1 part of the concentrated sample (10 parts trona, 1 part citric acid) and 10 parts distilled water—to form a trona-based sample. This trona-based sample was boiled for 3 minutes and then strained. The sample was tested about 15.75 months later, and subject to testing against five individual microorganisms provided in Table 10 in accordance with either the CTFA M-1 or CTFA M-2 guidelines. Pure culture tests yielded specific data on each microorganism employed in the study.

### Results:

TABLE 10
Testing of Trona-Based Sample

| Microorganism | Amount/Concentration | Method |
|---|---|---|
| Total Aerobic | <10 (CFU/g) | USP <2021> |
| Microbial Count | | |
| Coliform | <10 (CFU/g) | AOAC 991.14 |
| E. coli | Absent | USP <2022> |
| | (per 10 grams) | |
| S. Aureus | Absent | USP <2022> |
| | (per 10 grams) | |
| Salmonella spp. | Absent | USP <2022> |
| | (per 10 grams) | |
| Total Yeast and | <10 (CFU/g) | USP <2021> |
| Mold Count | | |
| Yeast | <10 (CFU/g) | USP <2021> |
| Mold | <10 (CFU/g) | USP <2021> |

The test results show that this trona-based sample prevented the growth of microorganisms after about 15.75 months.

### Example 11—Evaluation of Trona-Based Composition's Potential to Decrease Erythemal Response from UV Irradiation and Provide Remediation of the Skin to Future UC Radiation

### Objective:

To determine the ability of the trona-based composition to reduce redness (e.g., erythema) caused by UV irradiation and provide remediation of the skin to protect against future exposure to UV radiation.

### Inclusion Criteria:

Healthy male or female subjects:
a) 18-60 years of age;
b) Skin types I-III, determined by the following guidelines:
   I. Burns easily; rarely tans (sensitive);
   II. Burns easily; minimally tans (sensitive);
   III. Burns moderately; rarely gradually (normal);
   IV. Burns minimally; tans normally (normal);
   V. Burns rarely; tans profusely (insensitive);
   VI. Burns never; deeply pigmented (insensitive);
c) Absence of any visible skin disease which might be confused with a skin reaction from the test material;
d) Having completed a Medical History Form;
e) Having read, understood, and signed an Informed Consent Form;
f) Considered dependable and capable of following directions;
g) Willing to refrain from excessive sun exposure during the course of the study;

### Exclusion Criteria:

a) History of abnormal response to sunlight;

b) Subjects exhibiting current sunburn, suntan, uneven skin tone or visible skin disease which might interfere with evaluation of test results;

c) Pregnant or lactating females;

d) Subjects taking medication which might produce an abnormal response to sunlight or interfere with the results of the test.

### Test Method:

Ten (10) subjects who met the inclusion criteria were selected for participation. The subjects received each test material to the appropriate site with an additional site as an untreated irradiated control.

### Light Source:

A Xenon Arc Solar Simulator* (150w) was used as the source of ultraviolet light. A continuous emission spectrum in the UV range (290-400 nanometers) was utilized during the course of this testing procedure. The filters used in the Simulator were WG (1 mm) and UG 5 (1 mm).

### Colorimetry:

A tristimulus colorimeter (Smart Probe 400′) was used to measure erythema levels. The a* value of the L*a*b* color notation system is indicative of color changes in the red-green color axis. The higher the value, the more intensely red the object being evaluated. Therefore, the a* value was used as a measure of redness (erythema) on the skin surface, where an increase indicates an increase in erythema.

### Day 1:

The MED is defined as the time interval or dosage of UV light irradiation sufficient to produce a minimal, perceptible erythema on untreated skin. The MED of each subject was determined by a progressive sequence of timed UV light exposures, each of which is graduated incrementally by 25% over that of the previous exposure.

### Day 2:

Approximately sixteen (16) to twenty-four (24) hours after the MED irradiation, the sites were evaluated for erythema according to the following scoring system:
0=Negative, no visible response
0.5=Minimal erythema
1=Defined erythema
2=Moderate erythema
3=Severe erythema
   If there was a failure to elicit a MED response, the subject was disqualified.

A technician outline six (6) 1″×1.5″ test site areas on the subject's back between the scapulae and the belt-line, lateral to the mid-line. These areas were designated as the test sites. The test sites were exposed to 1.5× their pre-determined MED.

### Day 3:

Twenty-four (24) hours after irradiation, skin redness was evaluated visually by a trained technician using the scoring scale listed above. Baseline color readings were performed.

The test material was applied to five (5) of the six (6) test sites at the rate of 0.2 ml and covered with the gauze portion of an adhesive dressing to form an occluded patch. The remaining site was designated as an untreated irradiated control.

Approximately three (3) hours after product application, the patches were removed.

Four (4) hours after application, erythema was evaluated visually by a technician. Color readings were performed.

### Day 4:

Twenty-four (24) hours after test material application, skin redness was evaluated visually by a trained technician using the scoring scale listed above. Color readings were performed.

After evaluation of the test sites, each test site was exposed to UV radiation as listed below in Table 11-A:

TABLE 11-A

| Test Site | Radiation Dose |
|---|---|
| 1 | 0.25 × MED |
| 2 | 0.50 × MED |
| 3 | 0.75 × MED |
| 4 | 1.00 × MED |
| 5 | 1.50 × MED |
| 6 | NONE |

Immediately after irradiation, the test material was applied to five (5) irradiated test sites at the rate of 0.2 ml and covered with the gauze portion of an adhesive dressing to form an occluded patch.

Approximately three (3) hours after product application, the patches were removed.

Four (4) hours after application, erythema was evaluated visually by a technician. Color readings were performed.

### Statistics:

Colorimeter Data: The t-Test Dependent was used to determine if any significant differences (P<0.05) were observed in the mean a* values at each post-treatment scoring interval for the treated test sites compared to the untreated irradiated control.

Visual Evaluations: The t-Test Dependent was used to determine if any significant differences (P<0.05) were observed in the mean visual erythema scores at each post-treatment scoring interval for the treated test sites compared to the untreated irradiated control.

### Results:

Ten (10) subjects completed the study. Subject demographics are presented in Table 11-B. Appendix 11-I (Table 11-C-1 to Table 11-F-2) outlines each subject's mean a* value and visual erythema scores with the statistical comparison of the test material vs. the Untreated Irradiated Control at each evaluation interval.

### SUMMARY

Under the test conditions described, for the mean a* value, no significant differences were observed between the test material treated test sites and the untreated irradiated control test site at each evaluation interval.

On day 3 (4 hours after the first application) the visual erythema scores for test site 3 were significantly lower than the untreated irradiated control. This reduction indicates a reduction in erythema.

On day 4 (4 hours after the second irradiation/second application) no significant differences (mean a* value and visual erythema scores) were observed between the irradiated test material treated test sites and the untreated irradiated control. This suggests that the test material provided protection against the second UV exposure.

It was also noted that the subjects felt heat during the second irradiation of the test sites. When the test material was applied after irradiation, the subjects had a cooling sensation.

TABLE 11-B
Subject Data

| | | | | MED |
|---|---|---|---|---|
| Subject | Skin Type | Age | Sex | (seconds) |
| 1 | III | 39 | F | 15 |
| 2 | III | 39 | F | 19 |
| 3 | III | 30 | F | 15 |
| 4 | II | 41 | F | 19 |
| 5 | III | 49 | F | 24 |
| 6 | III | 27 | F | 25 |
| 7 | II | 46 | F | 12 |
| 8 | II | 56 | F | 12 |
| 9 | II | 33 | F | 15 |
| 10 | III | 43 | F | 24 |

Appendix 11-I: Mean a* values and Visual Erythema Scores

TABLE 11-C-1
Day 3 - Baseline to 24 hours after
irradiation - Color Meter - Mean a* Value

| | | | | | | Untreated |
|---|---|---|---|---|---|---|
| Subject | Site 1 | Site 2 | Site 3 | Site 4 | Site 5 | Control |
| 1 | 16.90 | 14.33 | 15.47 | 15.03 | 15.87 | 16.47 |
| 2 | 10.07 | 10.10 | 10.37 | 10.47 | 9.23 | 9.50 |
| 3 | 10.13 | 10.33 | 9.87 | 10.87 | 10.37 | 9.50 |
| 4 | 12.83 | 12.57 | 13.30 | 13.60 | 14.43 | 17.83 |
| 5 | 11.20 | 11.60 | 10.17 | 10.47 | 10.43 | 11.67 |
| 6 | 10.70 | 11.70 | 10.37 | 13.37 | 11.57 | 11.70 |
| 7 | 7.27 | 7.37 | 7.33 | 8.53 | 7.60 | 7.00 |
| 8 | 9.33 | 9.37 | 8.73 | 7.77 | 4.87 | 8.63 |
| 9 | 13.20 | 11.57 | 10.60 | 12.10 | 13.40 | 14.17 |
| 10 | 10.23 | 10.13 | 9.30 | 10.97 | 9.73 | 8.23 |
| Mean | 11.19 | 10.91 | 10.55 | 11.32 | 10.75 | 11.47 |

t-Test Dependent (Untreated control vs.)

| | | | | | | |
|---|---|---|---|---|---|---|
| t | 0.477 | 0.832 | 1.543 | 0.226 | 1.327 | N/A |
| df | 9 | 9 | 9 | 9 | 9 | N/A |
| Two- | 0.645 | 0.427 | 0.157 | 0.827 | 0.217 | N/A |
| tailed p< | | | | | | |
| r | 0.869 | 0.887 | 0.895 | 0.834 | 0.882 | N/A |

TABLE 11-C-2
Day 3 - Baseline to 24 hours after
irradiation - Visual Erythema Scores

| | | | | | | Untreated |
|---|---|---|---|---|---|---|
| Subject | Site 1 | Site 2 | Site 3 | Site 4 | Site 5 | Control |
| 1 | 2 | 2 | 2 | 2 | 2 | 2 |
| 2 | 2 | 2 | 2 | 2 | 2 | 2 |
| 3 | 1 | 1 | 1 | 1 | 1 | 1 |
| 4 | 1 | 1 | 1 | 1 | 1 | 1 |
| 5 | 1 | 1 | 1 | 1 | 1 | 1 |
| 6 | 1 | 1 | 1 | 1 | 1 | 1 |
| 7 | 0.5 | 0.5 | 0.5 | 0.5 | 0.5 | 0.5 |
| 8 | 2 | 2 | 2 | 2 | 0.5 | 2 |
| 9 | 2 | 2 | 2 | 2 | 2 | 2 |
| 10 | 0.5 | 0.5 | 0.5 | 0.5 | 0.5 | 0.5 |
| Mean | 1.30 | 1.30 | 1.30 | 1.30 | 1.30 | 1.30 |

t-Test Dependent (Untreated control vs.)

| | | | | | | |
|---|---|---|---|---|---|---|
| t | N/A | N/A | N/A | N/A | 1.000 | N/A |
| df | N/A | N/A | N/A | N/A | 9 | N/A |
| Two- | N/A | N/A | N/A | N/A | 0.343 | N/A |
| tailed p< | | | | | | |
| r | N/A | N/A | N/A | N/A | 0.716 | N/A |

TABLE 11-D-1
Day 3-4 Hours After First
Application - Color Meter - Mean a* Value

| | | | | | | Untreated |
|---|---|---|---|---|---|---|
| Subject | Site 1 | Site 2 | Site 3 | Site 4 | Site 5 | Control |
| 1 | 15.10 | 15.10 | 14.77 | 15.73 | 15.30 | 15.27 |
| 2 | 10.23 | 10.20 | 10.10 | 10.03 | 9.37 | 8.93 |
| 3 | 9.70 | 10.27 | 9.30 | 9.33 | 9.87 | 9.07 |
| 4 | 11.80 | 12.93 | 13.97 | 15.90 | 17.23 | 20.53 |
| 5 | 12.03 | 10.53 | 9.27 | 11.43 | 10.47 | 11.10 |
| 6 | 10.47 | 11.70 | 10.57 | 13.00 | 12.27 | 12.00 |
| 7 | 7.53 | 7.03 | 6.97 | 7.97 | 7.33 | 6.93 |
| 8 | 9.13 | 9.10 | 8.47 | 9.30 | 4.30 | 7.70 |
| 9 | 13.00 | 13.90 | 10.73 | 11.97 | 12.77 | 13.70 |
| 10 | 10.60 | 9.30 | 9.20 | 10.10 | 10.40 | 9.67 |
| Mean | 10.96 | 11.01 | 10.33 | 11.48 | 10.93 | 11.49 |

t-Test Dependent (Untreated control vs.)

| | | | | | | |
|---|---|---|---|---|---|---|
| t | 0.553 | 0.587 | 1.610 | 0.023 | 1.128 | N/A |
| df | 9 | 9 | 9 | 9 | 9 | N/A |
| Two- | 0.594 | 0.572 | 0.142 | 0.982 | 0.289 | N/A |
| tailed p< | | | | | | |
| r | 0.701 | 0.802 | 0.888 | 0.935 | 0.924 | N/A |

TABLE 11-D-2
Day 3-4 Hours After First Application - Visual Erythema Scores

| | | | | | | Untreated |
|---|---|---|---|---|---|---|
| Subject | Site 1 | Site 2 | Site 3 | Site 4 | Site 5 | Control |
| 1 | 2 | 2 | 2 | 2 | 2 | 2 |
| 2 | 2 | 2 | 1 | 2 | 1 | 2 |
| 3 | 1 | 1 | 1 | 1 | 1 | 1 |
| 4 | 1 | 0.5 | 1 | 2 | 2 | 2 |
| 5 | 1 | 1 | 0.5 | 1 | 1 | 1 |
| 6 | 1 | 1 | 0.5 | 1 | 0.5 | 1 |
| 7 | 0.5 | 0.5 | 0.5 | 0.5 | 0.5 | 0.5 |
| 8 | 2 | 2 | 2 | 2 | 0.5 | 2 |
| 9 | 2 | 1 | 1 | 2 | 2 | 2 |
| 10 | 0.5 | 0.5 | 0.5 | 0.5 | 0.5 | 0.5 |
| Mean | 1.30 | 1.15 | 1.00 | 1.40 | 1.10 | 1.40 |

t-Test Dependent (Untreated control vs.)

| | | | | | | |
|---|---|---|---|---|---|---|
| t | 1.000 | 1.464 | 2.753 | N/A | 1.765 | N/A |
| df | 9 | 9 | 9 | N/A | 9 | N/A |
| Two- | 0.343 | 0.177 | 0.022 | N/A | 0.111 | N/A |
| tailed p< | | | | | | |
| r | 0.881 | 0.647 | 0.731 | N/A | 0.677 | N/A |

TABLE 11-E-1
Day 4-24 Hours After First
Application - Color Meter - Mean a* Value

| | | | | | | Untreated |
|---|---|---|---|---|---|---|
| Subject | Site 1 | Site 2 | Site 3 | Site 4 | Site 5 | Control |
| 1 | 17.27 | 17.83 | 17.53 | 16.57 | 16.23 | 15.37 |
| 2 | 11.33 | 10.57 | 9.77 | 8.70 | 9.00 | 8.43 |
| 3 | 9.47 | 10.33 | 9.90 | 9.77 | 10.17 | 9.40 |
| 4 | 11.03 | 11.23 | 13.63 | 13.83 | 14.43 | 18.83 |
| 5 | 12.20 | 11.33 | 10.63 | 11.83 | 9.47 | 10.23 |
| 6 | 10.67 | 10.60 | 10.83 | 12.43 | 10.47 | 10.73 |
| 7 | 7.23 | 5.47 | 5.13 | 6.93 | 5.67 | 5.13 |
| 8 | 8.07 | 8.63 | 7.83 | 9.67 | 4.73 | 7.03 |
| 9 | 12.97 | 12.07 | 7.97 | 11.70 | 12.53 | 12.73 |
| 10 | 12.03 | 10.60 | 8.77 | 11.33 | 10.40 | 9.13 |
| Mean | 11.23 | 10.87 | 10.20 | 11.28 | 10.31 | 10.70 |

t-Test Dependent (Untreated control vs.)

| | | | | | | |
|---|---|---|---|---|---|---|
| t | 0.529 | 0.178 | 0.644 | 0.812 | 0.714 | N/A |
| df | 9 | 9 | 9 | 9 | 9 | N/A |
| Two- | 0.609 | 0.862 | 0.535 | 0.438 | 0.494 | N/A |
| tailed p< | | | | | | |
| r | 0.634 | 0.698 | 0.792 | 0.850 | 0.902 | N/A |

TABLE 11-E-2
Day 4 - 24 Hours After First Application - Visual Erythema

| | | | | | | Untreated |
|---|---|---|---|---|---|---|
| Subject | Site 1 | Site 2 | Site 3 | Site 4 | Site 5 | Control |
| 1 | 2 | 2 | 2 | 2 | 2 | 2 |
| 2 | 2 | 2 | 1 | 2 | 1 | 1 |
| 3 | 1 | 0.5 | 1 | 1 | 1 | 1 |
| 4 | 0.5 | 0.5 | 1 | 2 | 2 | 2 |
| 5 | 1 | 1 | 1 | 1 | 0.5 | 1 |
| 6 | 1 | 1 | 0.5 | 1 | 0.5 | 1 |
| 7 | 0.5 | 0.5 | 0.5 | 0.5 | 0.5 | 0.5 |
| 8 | 1 | 1 | 1 | 2 | 0.5 | 1 |
| 9 | 2 | 2 | 1 | 2 | 2 | 2 |
| 10 | 0.5 | 0.5 | 0.5 | 0.5 | 0.5 | 0.5 |
| Mean | 1.15 | 1.10 | 0.95 | 1.40 | 1.05 | 1.20 |

t-Test Dependent (Untreated control vs.)

| | | | | | | |
|---|---|---|---|---|---|---|
| t | 0.264 | 0.514 | 1.861 | 1.500 | 1.964 | N/A |
| df | 9 | 9 | 9 | 9 | 9 | N/A |
| Two- | 0.798 | 0.619 | 0.096 | 0.168 | 0.081 | N/A |
| tailed p< | | | | | | |
| r | 0.514 | 0.518 | 0.692 | 0.777 | 0.939 | N/A |

TABLE 11-F-1
Day 4-4 Hours After Second Irradiation/
Application - Color Meter - Mean a* Value

| | | | | | | Untreated |
|---|---|---|---|---|---|---|
| Subject | Site 1 | Site 2 | Site 3 | Site 4 | Site 5 | Control |
| 1 | 17.13 | 16.53 | 17.73 | 17.50 | 17.23 | 14.23 |
| 2 | 8.70 | 7.20 | 9.30 | 9.23 | 9.77 | 9.23 |
| 3 | 11.07 | 9.97 | 9.87 | 10.23 | 10.87 | 9.77 |
| 4 | 12.40 | 13.20 | 13.77 | 13.80 | 15.47 | 20.50 |
| 5 | 11.13 | 10.93 | 10.47 | 12.07 | 10.23 | 10.27 |
| 6 | 10.30 | 9.43 | 9.37 | 12.00 | 10.67 | 11.03 |
| 7 | 7.07 | 5.70 | 5.70 | 7.33 | 6.73 | 6.40 |
| 8 | 9.73 | 7.53 | 7.40 | 10.17 | 5.13 | 7.20 |
| 9 | 14.33 | 13.37 | 9.53 | 14.20 | 16.17 | 13.73 |
| 10 | 11.37 | 10.50 | 9.80 | 11.20 | 11.13 | 9.20 |
| Mean | 11.32 | 10.44 | 10.29 | 11.77 | 11.34 | 11.16 |

t-Test Dependent (Untreated control vs.)

| | | | | | | |
|---|---|---|---|---|---|---|
| t | 0.168 | 0.861 | 0.969 | 0.699 | 0.247 | N/A |
| df | 9 | 9 | 9 | 9 | 9 | N/A |
| Two- | 0.646 | 0.412 | 0.358 | 0.502 | 0.811 | N/A |
| tailed p< | | | | | | |
| r | 0.646 | 0.766 | 0.732 | 0.735 | 0.830 | N/A |

TABLE 11-F-2
Day 4-4 Hours After Second Irradiation/
Application - Visual Erythema

| | | | | | | Untreated |
|---|---|---|---|---|---|---|
| Subject | Site 1 | Site 2 | Site 3 | Site 4 | Site 5 | Control |
| 1 | 2 | 2 | 2 | 2 | 2 | 2 |
| 2 | 2 | 1 | 1 | 2 | 1 | 1 |
| 3 | 2 | 2 | 2 | 2 | 2 | 2 |
| 4 | 1 | 1 | 1 | 2 | 2 | 2 |
| 5 | 1 | 1 | 0.5 | 1 | 1 | 1 |
| 6 | 0.5 | 0.5 | 0.5 | 1 | 1 | 1 |
| 7 | 0.5 | 0.5 | 0.5 | 0.5 | 0.5 | 0.5 |
| 8 | 2 | 1 | 2 | 2 | 0.5 | 1 |
| 9 | 1 | 2 | 1 | 2 | 2 | 2 |
| 10 | 1 | 1 | 1 | 1 | 1 | 0.5 |
| Mean | 1.30 | 1.20 | 1.15 | 1.55 | 1.30 | 1.30 |

t-Test Dependent (Untreated control vs.)

| | | | | | | |
|---|---|---|---|---|---|---|
| t | 0.000 | 0.802 | 0.758 | 1.861 | 0.000 | N/A |
| df | 9 | 9 | 9 | 9 | 9 | N/A |
| Two- | 1.000 | 0.443 | 0.468 | 0.096 | 1.000 | N/A |
| tailed p< | | | | | | |
| r | 0.375 | 0.793 | 0.505 | 0.763 | 0.931 | N/A |

### Example 12—Treatment of Burns

Subjects were provided with the trona-based spray for treating burns. Subject were directed to: (i) Spray affected area until skin surface is visibly wet, and (ii) Apply spray as often as needed to keep the dermis cooled. The results are provided in Table 12.

TABLE 12

| | | | | Bum | Bum | Bum | % of Body | Pain pre- | Pain post- |
|---|---|---|---|---|---|---|---|---|---|
| Patient | Gender | Age | Skin Color | Location | Source | severity | Burned | treatment | treatment |
| 1 | M | 30 | 3 | Left calf | Chemical | 3 | 0-10% | 5 | 4 |
| 2 | F | 24 | — | Neck | Laser | 2 | 0-10% | 5 | 2 |
| 3 | M | 25 | 2 | Palm | Fire | 1.5 | 0-10% | 3 | 2 |
| 4 | F | 40 | 1 | Face | IPL | 2 | 0-10% | 2 | 1 |
| | | | | | machine | | | | |

### Example 13—Treatment of Burns

### Directions:

In each, case, the spray or ointment was directed to be applied as follows:
1. Spray:
   a. Spray affected area until skin surface is visibly wet.
   b. Apply spray as often as needed to keep dermis cooled.
   c. Heat and pain should decrease within the first 12-24 hours.
2. Cream:
   a. Being using the cream after 12-24 hours when dermis tenderness is such that the cream can be applied by the patient.
   b. Apply cream lightly and without rubbing in completely. Leave a thin layer of the cream on the skin's surface.
   c. Continue to apply cream every 4 hours for the next few days.
   d. Skin should being to heal and the redness should be gone within a few days.
      Case 13-1A: Burn from boiling water

A young woman was admitted to the ER after spilling boiling water on her hand. The young woman had severe pain with redness. The burn was confirmed as being a first degree burn. The young woman sprayed her hand with the trona-based spray (Example 1-B and Example 23). The pain on her hand was greatly reduced within a minute of application.

Case 13-1B: Burn from hot grease

A man had splattered hot grease on his arm while cooking. The man sprayed his hand with the trona-based spray (Example 1-B and Example 23). The spray provided immediate pain relief. The man recovered from the burns on his arm without any scarring.

Case 13-1C: Burn from contact

A woman was cooking fried chicken and was severely burned on the inside of her wrist. The woman ran cold water over the burn for a few minutes but could still feel that the burn was spreading. After several hours, the burn was approximately 2 inches by 2 inches and blistering.

Two days later, the woman was provided with a bottle of the trona-based spray (Example 1-B and Example 23). Within one minute of spraying the affected area with the spray, the burning sensation was gone. The woman applied the spray to the affected area 3-4 times during the day and noticed that the appearance and the sensation of the burn had increased from before.

After several days of applying the spray, the woman noticed that the burn was healing without scarring. After a few weeks, the burn was barely visible on her wrist.

### Case 13-2A: Sunburn

A young female got a sunburn with blisters. The young adult used the trona-based ointment (Example 1-B and Example 24). Within 2 minutes of application, the pain was gone.

### Case 13-2B: Sunburn

A young woman got a sunburn after more than 4 hours outside on a lake. The young woman used the trona-based ointment (Example 1-B and Example 24). Within 2 minutes of application, the pain and related discomfort was gone.

### Case 13-2C: Sunburn

I young woman got a sunburn after a day of water skiing. The trona-based spray was applied to the young woman and provided pain relief.

### Case 13-2D: Sunburn

I young man got a sunburn after a day of water skiing. The young man was nauseated and vomited with chills. The trona-based spray was applied to the young man 3-4 times over the course of 4 hours and the symptoms subsided.

### Case 13-2E: Sunburn

I boy got a sunburn after a day of water skiing. The young man was nauseated and had pain from the sunburn. The trona-based spray was applied to the young man and the symptoms subsided.

### Case 13-2F: Sunburn

A woman got a sunburn after a day of water skiing. At first, she felt slight discomfort from the sunburn. A few hours later, the woman had severe pain with chills. The affected area was sprayed with the trona-based spray and provided instant pain relief After about 5 minutes, the pain returned, and the spray was re-applied and the pain subsided again. The woman continued to apply the spray over 2-3 days with a instant reduction in pain with each application.

### Case 13-2G: Sunburn

A man got sunburnt after a day of water skiing. After a few hours, the man had pain in his shoulders and back. The affected area was sprayed with the trona-based spray as needed over the course of a few days. The man had blisters the day after first getting sunburnt but within a week, the blisters were gone without any scarring.

### Case 13-2H: Sunburn

A woman applied the trona-based spray before going outside and did not get a sunburn. On a different occasion, the woman went outside without having applied the trona-based spray and got a sunburn. She applied the trona-based spray to the affected area and the pain was gone within 24 hours.

### Case 13-21. Sunburn

A man got a sunburn and applied the trona-based ointment and spray to relieve the pain. The application of the trona-based ointment and spray cut the expected healing time in half compared to not using the trona-based ointment and spray.

### Case 13-2J. Sunburn

A woman got a sunburn after about 20 minutes even with sunscreen applied. The trona-based spray was applied to the affected area a few times per day and within two days the pain was completely gone.

Case 13-3A: Burn from direct contact with stove

A woman burned herself on the stove while she was cooking. The woman immediately applied the trona-based spray to the affected area. The redness, swelling, and pain subsided immediately. The affected area healed on an accelerated timeline without any scarring.

Case 13-3B: Burn from direct contact with curling iron.

A young woman burned herself using a curling iron. The woman immediately applied the trona-based spray to the affected area 2-3 times over the course of a day. The application provided instant relief and within one day the affected area was healed without any scarring.

### Example 14—Treatment for Itching

### Case 14A: Itching

A man experienced a blood clot in his leg which caused the leg to swell. The clot was treated to reduce the swelling but resulted in extremely irritating itching. The man sprayed his leg with the trona-based spray (Example 1-B and Example 23). This application of spray resulted in immediate itch relief. When the effects of the the spray wore off, the man applied more spray to the affected area which resulted in further itch relief

### Case 14B: Itching

A man had severe itching as a result of kidney dialysis. The man applied the trona-based cream to the affected area which provided instant itching relief.

### Example 15—Treatment for Poisonous Plant Contact

### Case 15A: Poisonous Plants

A woman was in direct contact with poison oak on her neck and upper arm. The woman immediately applied the trona-based spray to the affected area. The pain and itching was relieved. The woman further applied the trona-based spray to the affected area a 1-2 times per day over the course of a 2-3 days The affected area healed without any scarring and the skin stayed soft and pliable.

Case 15B: Rash from Poisonous Plants

A woman got a rash after coming into contact with some poisonous plants. The rash was bumpy, sore, and itchy. After applying the trona-based cream to the affected area, the ich and soreness was alleviated instantly. The woman applied the trona-based cream 2-3 times per day over 2-3 days which resulted in the rash being removed.

Case 15C: Rash from Poisonous Plants

A young girl went camping with her family and was in contact with some poisonous plants. The contact resulted in bumps and redness on her arms with extreme itchiness and pain. The trona-based spray was applied to her arm and the itchiness and pain resolved within a few hours.

Case 15D: Rash from Poisonous Plants

A professional gardener came across a poisonous plant and ended up with a rash. The subject applied the trona-based spray to her rash which resulted in instant relief from discomfort associated with the rash. After a few hours, the rash was gone.

Case 15E: Rash from Poisonous Plants

A woman contacted a poisonous plant which resulted in a large rash on her chest. The rash was about 3 inches in diameter with redness, stinging, and burning pain. The woman applied the trona-based cream to the affected area twice per day over the course of seven days. After three days, the rash had changed from the redish color and itched substantially less. After a week, the rash, redness, stinging, and burning pain were completely gone.

### Example 16—Treatment for Wounds

### Case 16A: Wound-Healing, Peripheral Neuropathy Relief, Reduction in Post-Workout Muscle Soreness

Numerous patients (>10) were seen in a health clinic over the course of 18 months. The trona-based spray and trona-based ointment were both used. The trona-based spray and trona-based ointment were found to reduce discomfort associated with peripheral neuropathy, reduce post-workout muscle soreness, and heal wounds. The trona-based spray and trona-based ointment reduced pain while promoting dilation of the small blood vessels to enhance circulation and enhance the delivery of nutrients to and elimination of waste from cells.

### Case 16B: Wound-Healing

A man got a gash on his head after a bungee strap hit him above his right eye. The metal hook caused a cut that was about ¼ inch in length. The man washed off the cut and applied the trona-based spray to the affected area. The swelling and bleeding stopped without any scabbing or scarring.

### Example 17—Treatment Insect Bites and Bee Stings

Case 17A: Insect bites

A young girl got a mosquito bite. She applied the trona-based spray to the affected area. The itching and swelling was instantly reduced upon application.

Case 17B: Bee stings

A man was stung by a bee which resulted in pain and swelling at the stung area. The man applied the trona-based spray to the affected area. The pain and swelling were reduced within a few minutes to be unnoticeable.

Case 17C: Bee stings

A man in bee gear was stung three times in a gap between his ankle and the bee gear. The man applied the trona-based spray to the affected area. The pain and swelling was reduced within a few minutes.

Case 17D: Ant bites

A man was bitten by numerous ants from an ant hill which resulted in copious itching. The man applied the trona-based cream to the affected area and felt instant itching relief.

### Example 18—Treatment for Shingles (Herpes Zoster Lesions)

### Case 18: Shingles

A woman had an outbreak of shingle lesions on her thighs. After a few days, the small blisters on her thighs were extremely painful and burning. The woman applied the trona-based spray to the affected areas on her thighs. Within 10 seconds of application, the woman had relief from the burning and the pain.

### Example 19—Treatment for Neuropathy

### Case 19A: Neuropathy

Numerous infected and diabetic patients with neuropathy. The trona-based cream was applied to the patients. The localized pain and uncomfortable neuropathies (e.g., tingling and discomfort) were relieved by the instantaneous and continued application of the cream.

### Case 19B: Neuropathy and Edema

A woman applied the trona-based cream daily over the course of 6 months. Before using the trona-based cream, the woman would have painful swelling in her feet and legs. The daily application of the trona-based cream resulted in the elimination of the painful swelling in her feet and legs. After 6 months, the woman ceased application of the trona-based cream which resulted in the return of the painful swelling in her feet and legs. The woman recommenced daily use of the trona-based cream which once again resulted in the elimination of the painful swelling in her feet and legs.

### Example 20—Treatment for Acne

### Case 20: Acne

A woman had acne that had begun to redden and sting. The woman applied the trona-based cream to her acne which resulted in instant pain relief. The woman used the trona-based cream on the acne over the course of two weeks. The woman's complexion was substantially free of acne after the two weeks of using the trona-based cream.

### Example 21—Treatment for Venous Insufficiency

### Case 21: Venous Insufficiency and Range of Motion

A woman with venous insufficiency disease in her left leg, ankle, and foot used the trona-based cream. The black veins in her left leg, ankle, and foot changed to pink within a few days of treatment. The woman also had limited motion in her left shoulder due to surgery. After applying the trona-based cream to her left shoulder, the woman's range of motion in her left shoulder was enhanced in comparison to the range of motion before she had applied the trona-based cream.

### Example 22—Treatment for Muscle Soreness and Edema

Case 22: Muscle Soreness

A woman applied the trona-based cream to relax her muscles, reduce soreness, and facilitate a chiropractic adjustment. The application of the trona-based cream resulted in substantial muscle relaxation.

### Example 23—Trona-Based Spray

A trona-based spray was prepared as shown in Table 23.

TABLE 23
Trona based spray

| Ingredient | Percent (w/v) | Amount | |
|---|---|---|---|
| Trona (in 90% distilled water) | 10% | 60 | ml |
| Humectant (e.g., propylene glycol) | | 300 | mg |
| Aloe Vera 1x | | 1 | mg |
| Preservative (e.g., benzalkonium chloride) | | 6 | mg |

### Example 24—Trona-Based Cream

A trona-based cream was prepared as shown in Table 24.

TABLE 24
Trona based cream

| Ingredient | Percent (w/v) | Amount | |
|---|---|---|---|
| Trona (in 90% w/v distilled water) | 10% | 600 | ml |
| Humectant (e.g., glyceine DSP) | | 100 | mg |
| Aloe Vera 1x | | 1 | mg |
| Preservative (e.g., benzalkonium chloride) | | 6 | mg |
| Avocado Oil | | 75 | mg |

The trona-based cream can comprise appropriate amounts of deionized water, cetyl alcohol, glyceryl monostearate, isopropyl myristate, stearyl alcohol, avocado oil, sunflower oil, propylene glycol, glycerine, Vitamin E, lipoic acid, sodium benzoate, potassium sorbate, urea USP polysorbate 20, xanthan gum, appropriate fragrances, and methylsulfonylmethane.

The foregoing detailed description describes the disclosure with reference to specific exemplary embodiments. However, it will be appreciated that various modifications and changes can be made without departing from the scope of the present disclosure as set forth in the appended claims. The detailed description and accompanying drawings are to be regarded as merely illustrative, rather than as restrictive, and all such modifications or changes, if any, are intended to fall within the scope of the present disclosure as described and set forth herein.

What is claimed is:

1. A method of treating a topical ailment, comprising:
a) identifying the topical ailment at a topical region of a subject;
b) procuring a trona based therapeutic composition including a sodium carbonate mineral and a carboxylic acid, wherein the therapeutic composition is sterilized; and
c) administering the therapeutic composition to the topical region of the subject.

2. The method of claim 1, further comprising:
sterilizing the therapeutic composition by heating at a temperature greater than a boiling point of the trona based therapeutic composition and for a duration of from about 7 minutes to about 20 minutes to destroy substantially all bacteria and form a sterilized mixture.

3. The method of claim 1, wherein the therapeutic composition is sterilized when the therapeutic composition passes a preservative efficacy test comprising at least one of: a United States Pharmacopeia (USP)<61> Microbial Limits Test, a Personal Care Products Council (PCPC) M-1 “Determination of the Microbial Content of Cosmetic Products” test, a PCPC M-2 “Examination of Staphylococcus aureus, Escherichia coli and Pseudomonas aeruginosa” test, a PCPC M-3 “A Method for Preservation Testing of Water-Miscible Personal Care Products,” a PCPC M-4 “Method for Preservation Testing of Eye Area Cosmetics” test, a USP <2021>“Microbial Enumeration” test, a USP <2022>“Absence of Specified Microorganisms” test, an AOAC Official Method 991.14 “Coliform and Escherichia coli Counts in Foods” test, and a combination thereof.

4. The method of claim 1, wherein the ailment is one of more of fire burns, sunburns, abrasions, wounds, medical burns, insect bite, insect sting, skin cut, cold sore, itch, rash, poisonous plant exposure, mouth irritation, warts, corns, razor abrasions, athletes foot, nail fungus, cuticle infection, dry skin, eczema, psoriasis, scalp irritation, sores, fever, peripheral neuropathy, neuropathy, edema, erythema, pain, scarring, venous insufficiency disease, muscle soreness, and disinfection.

5. The method of claim 1, wherein the ailment is fever.

6. The method of claim 1, wherein the sodium carbonate mineral is a member selected from the group consisting of trona, gaylussite, natron, prissonite, northupite, nahcolite, thermonatrite, and combinations thereof.

7. The method of claim 1, wherein the sodium carbonate mineral is trona.

8. The method of claim 1, wherein the carboxylic acid is a mono-, di- or tricarboxylic acid.

9. The method of claim 1, wherein the carboxylic acid is one or more of citric acid and salicylic acid.

10. The method of claim 1, wherein the carboxylic acid consists essentially of citric acid.

11. The method of claim 1, wherein the carboxylic acid is citric acid and the sodium carbonate mineral is trona and a w/w ratio of trona to citric acid is 200:1 to 5:1.

12. The method of claim 1, wherein the administering includes selecting a dosage sufficient to produce a therapeutic effect on the ailment and contacting the topical region.

13. The method of claim 12, wherein the dosage comprises 1 to 5 topical squirts, pumps, sprays, or a combination thereof.

14. A trona based therapeutic device, comprising a trona based therapeutic composition contained in a structural carrier, wherein the therapeutic composition is sterilized and includes a sodium carbonate mineral and a carboxylic acid.

15. The device of claim 14, wherein the therapeutic composition is sterilized when the therapeutic composition passes a preservative efficacy test comprising at least one of: a United States Pharmacopeia (USP)<61> Microbial Limits Test, a Personal Care Products Council (PCPC) M-1 “Determination of the Microbial Content of Cosmetic Products” test, a PCPC M-2 “Examination of Staphylococcus aureus, Escherichia coli and Pseudomonas aeruginosa” test, a PCPC M-3 “A Method for Preservation Testing of Water-Miscible Personal Care Products,” a PCPC M-4 “Method for Preservation Testing of Eye Area Cosmetics” test, a USP <2021>“Microbial Enumeration” test, a USP <2022>“Absence of Specified Microorganisms” test, an AOAC Official Method 991.14 “Coliform and Escherichia coli Counts in Foods” test, and a combination thereof.

16. The device of claim 14, wherein the sodium carbonate mineral is a member selected from the group consisting of trona, gaylussite, natron, prissonite, northupite, nahcolite, thermonatrite, and combinations thereof.

17. The device of claim 14, wherein the carboxylic acid is one or more of citric acid and salicylic acid.

18. The device of claim 14, wherein the carboxylic acid is citric acid and the sodium carbonate mineral is trona and a w/w ratio of trona to citric acid is 200:1 to 5:1.

19. The device of claim 14, wherein the carrier is selected from the group consisting of a fabric wipe, a compress, and a bandage.

20. A method of making a trona based therapeutic composition comprising:
mixing a sodium carbonate mineral and a carboxylic acid to form a precursor mixture;
heating the precursor mixture at a temperature and for a time sufficient to destroy substantially all bacteria and form a sterilized mixture; and
filtering the sterilized mixture to form a sterilized therapeutic composition.

21. The method of claim 20, wherein the time is from 7 to 20 minutes and the temperature is above a boiling point of the precursor mixture.

22. The method of claim 20, wherein the therapeutic composition is sterilized when the therapeutic composition passes a preservative efficacy test comprising at least one of: a United States Pharmacopeia (USP)<61> Microbial Limits Test, a Personal Care Products Council (PCPC) M-1 “Determination of the Microbial Content of Cosmetic Products” test, a PCPC M-2 “Examination of Staphylococcus aureus, Escherichia coli and Pseudomonas aeruginosa” test, a PCPC M-3 “A Method for Preservation Testing of Water-Miscible Personal Care Products,” a PCPC M-4 “Method for Preservation Testing of Eye Area Cosmetics” test, a USP <2021>“Microbial Enumeration” test, a USP <2022>“Absence of Specified Microorganisms” test, an AOAC Official Method 991.14 “Coliform and Escherichia coli Counts in Foods” test, and a combination thereof.

23. The method of claim 20, wherein the sodium carbonate mineral is a member selected from the group consisting of trona, gaylussite, natron, prissonite, northupite, nahcolite, thermonatrite, and combinations thereof.

24. The method of claim 20, wherein the carboxylic acid is one or more of citric acid and salicylic acid.

25. The method of claim 20, wherein the carboxylic acid is citric acid and the sodium carbonate mineral is trona and a w/w ratio of trona to citric acid is 200:1 to 5:1.
